(19) United States
(12) Patent Application Publication
(10) Pub. No.: US 2006/0187068 A1
(43) Pub. Date: Aug. 24, 2006

(54) Emergency situation detector

(76) Inventor: David Cohen, Tel-Aviv (IL)

Correspondence Address: Martin Moynihan;Prtsi Inc, PO Box 16446, Arlington, VA, 22215, US

(21) Appl. No.: 10/565,552

(22) PCT Filed: Mar. 29, 2004
(86) PCT No.: PCT/IL04/00288

§ 371 Date: Jan. 23, 2006

(30) Foreign Application Priority Data
Sep. 15, 2003 (IL) 157925
Mar. 8, 2004 (IL) 160783

(51) Int. Cl. G08B 23/00

(52) U.S. Cl. 340/576

(57) ABSTRACT

Emergency situation detection apparatus (12) comprises: a stress input unit for receiving body stress information from a subject (10), a physical input unit for receiving body physical reaction data (14) from said subject (10), a comparator unit (20), associated with said stress input unit and said physical input unit, for comparing stress level information and physical reaction data, to detect substantially simultaneous stress level change and a physical reaction in said subject (10), said apparatus being operable to threshold said simultaneous detection to infer the presence of an emergency situation and to enter an alarm state.

### FIELD OF THE INVENTION

The present invention relates to an emergency situation detector.

### BACKGROUND OF THE INVENTION

It is desirable to know when personnel encounter emergency situations. In particular security personnel including night watchman and guards, airline pilots, truck and van drivers and the like can be the subject of attacks and other emergencies with which they are unable to cope. In such a case it is desirable for the subject of the attack to call for help, but sometimes the nature of the emergency renders calling for help impossible. Likewise, elderly and other vulnerable persons, particularly those living on their own, can find themselves in difficulties and unable to reach a telephone to call for help, for example after a fall.

In cases where it is not possible to call for help, a number of systems exist for automatically determining that an emergency situation exists and calling for help.

Hospital-based systems that monitor a patient's pulse and call a doctor or nurse if the pulse falls are well known but are not suitable for anything other than the hospital environment.

Aircraft based hijack warning systems rely upon the pilot's standard radio-based voice link to air traffic control or include panic buttons for broadcasting an SOS signal. Hijackers however tend to be familiar with the presence of these systems and either use them to their advantage or prevent their use altogether.

Other systems for protecting aircraft from emergencies tend to rely on pilots' reaction times. Certain types of emergencies happen too quickly for the pilots to be able to raise the alarm or divert the pilots to emergency activity without diverting their attention to raising the alarm.

Often, the ability to determine what has happened following an aviation disaster is dependent on finding the aircraft flight recorder or black box.

Israel Patent Application No. 145498 to the present applicant discloses a system for detecting cockpit emergencies comprising the following:
a) an input unit for receiving body stress level information from at least two subjects,
b) a detection unit, associated with said input unit, for comparing stress level information from said at least two subjects, to detect substantially simultaneous stress level increases in said subjects,
the system being operable to threshold detected simultaneous stress level increases to infer the presence of an emergency situation and to enter an alarm state.

The system uses the physiological state of the pilots to determine that an emergency situation has arisen. In order to reduce false alarms it takes data from the two pilots and deduces the presence of an alarm when both pilots indicate stress. Such a system has the disadvantage that it is only useful in situations such as the cockpit of a civil aircraft where two or more persons are likely to undergo the same emergency. The system is not applicable to security guards, elderly people living alone and the like. Likewise it is not applicable for monitoring of persons being sent into dangerous situations such as troops into battle or firemen into a burning building.

### SUMMARY OF THE INVENTION

According to the present invention there is provided emergency situation detection apparatus comprising:

a stress input unit for receiving body stress level information from a subjects,

a physical input unit for receiving body physical reaction data from said subject.

a comparator unit, associated with said stress input unit and said physical input unit, for comparing stress level information and physical reaction data, to detect substantially simultaneous stress level increases and a physical reaction in said subject,

said apparatus being operable to threshold said simultaneous detection to infer the presence of an emergency situation and to enter an alarm state.

### BRIEF DESCRIPTION OF THE DRAWINGS

For a better understanding of the invention and to show how the same may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings,

FIG. 1 is a simplified diagram of a detection device according to a first embodiment of the present invention;

FIG. 2 is a simplified diagram showing the detection device of FIG. 1 in greater detail; and

FIG. 3 is a simplified diagram showing a 3d virtual figure for providing an intuitive user front end for monitoring the state of a subject or alternatively for providing a way of translating motion of the subject into an animation for a virtual reality game or an animated film or the like.

### DESCRIPTION OF THE PREFERRED EMBODIMENTS

The present embodiments provide an emergency situation detector which uses the fight or flight physiological response of subjects to determine that an emergency situation exists and to automatically raise an alarm. A supporting signal is then taken from an independent device which measures something other than body stress, such as physical body attitude. The use of an average of the signals from the stress and the physical detector provides protection against false alarms caused by self-induced anger, pure fright unaccompanied by an attack, and the like to which individual subjects may be susceptible. The signals may be measured against a threshold, or a delta may be used.

In a broader sense the present embodiments provide indications of dangerous situations arising or of circumstances that could lead to dangerous situations. For example, the embodiments may be able to from physiological measurements that a security guard has fallen asleep, and therefore is not doing his duty of guarding.

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

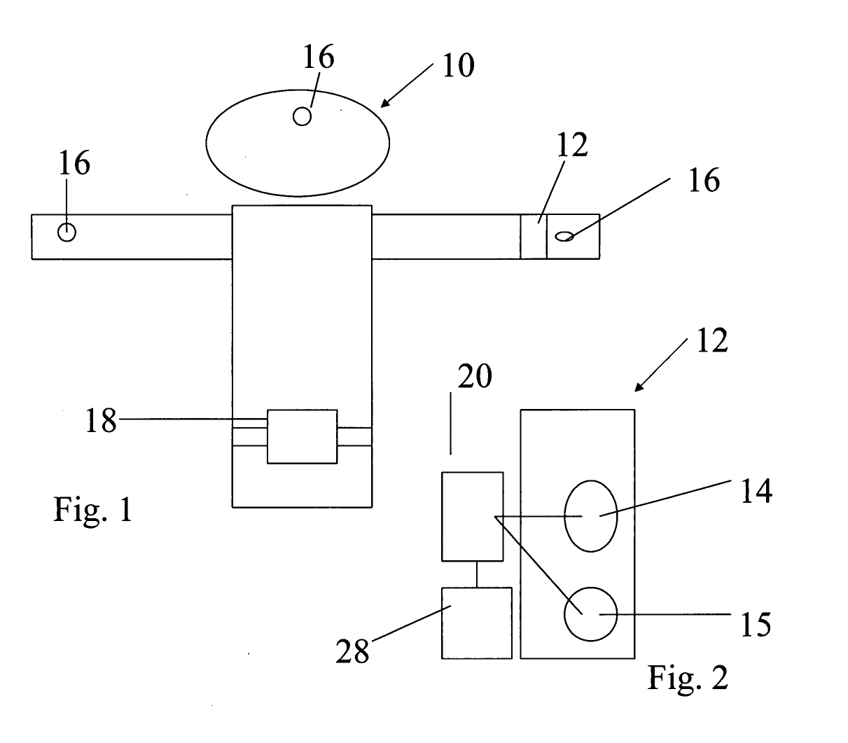

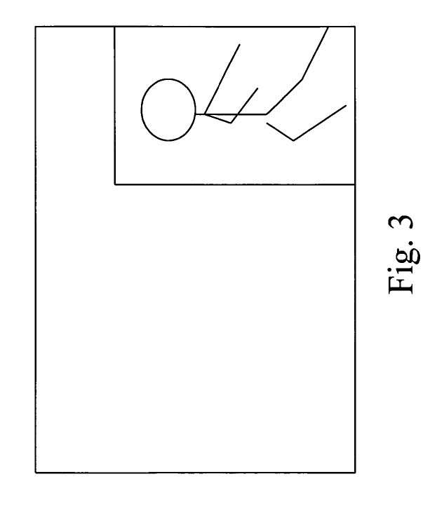

Reference is now made to FIG. 1, which shows an emergency situation detection apparatus placed on a user.

In FIG. 1, a subject 10 has an emergency situation detector 12 attached thereto. The detector 12 comprises bodily function detector 14 and physical reaction detector 15. The bodily function detector may for example detect pulse rate or sweat levels of the subject. Preferably the detector may be concealed beneath the subject's clothing. The detector 12 is preferably able to send signals in non-contact manner to emergency situation detection apparatus 16.

The bodily function detector 14 receives physiological body stress level information. The physical reaction detector preferably detects sudden movements, or indications of an impact of some kind or the attainment of a horizontal position or like indicators of physical reaction. An impact detector may for example comprise a piezoelectric sensor. Neither physiological stress alone nor physical reaction alone are reliable indicators of an emergency situation but both taken together may be expected to increase the reliability of any determination of an emergency situation.

Preferably a comparison unit 20 is associated with the bodily function and reaction detectors, for comparing signal information to determine substantially simultaneous occurrence of stress level increase and physical reaction in the subject. The detections may be thresholded as deemed sensible by the skilled person to increase reliability of detection. Different thresholds may be appropriate for different kinds of subject. Thus elderly people may be better served by a lower threshold than a security guard. As a further alternative, instead of a fixed threshold level, the system may monitor the change in signal level over time. The change or delta may then be thresholded. Thresholding the deltas can distinguish high signal levels which are due to a rapidly occurring event from high signal levels which may be due to background stress and the like.

The thresholded output of the comparison unit, following a positive result of the thresholding, is passed to an alarm state manager 28 to imply the presence of an emergency situation and to enter an alarm state.

Preferably the alarm state manager is able to call for assistance, for example via automatic opening of a radio link, or of a video link, to a central controller, thus to provide immediate indication of an emergency state. Preferably, the link, which is at least an audio link, includes at one end a speaker and or microphone located on the body of the user.

In a further preferred embodiment specifically for an aircraft cockpit, the alarm state manager is able to initiate an automatic download of the aircraft's flight recorder or black box data to a central controller, thus making available flight information even if the black box is never recovered.

The alarm state manager is preferably also able to enter an alarm state under the influence of other detectors, for example with detection of a loud noise or following prolonged instability. The alarm state manager may be able to enter different levels of alarm states prompting different actions.

In a further preferred embodiment of the present invention, the emergency situation detector includes an audio or other confirmation channel which can be opened upon detection of an emergency in order to provide confirmation of the situation or allow two-way communication, or the like.

In a further preferred embodiment the emergency situation detector 12 includes a GPS detector to provide positioning information. For use in a building or other places where GPS signals may not be available, a triangulation system may be installed for accurate positional information.

A further preferred embodiment intended for a user who stays within a predefined area, such as a security guard on patrol, simply sends regular code signals from which the system infers that he is in position.

Further preferred embodiments are provided to determine attitude, position and motion of a subject. Thus the emergency situation detector may include an accelerometer. A detector for detection of a direction that a user is facing may be strapped to the chest or a like part of the body. The detector includes a compass needle and the relative alignment of the compass needle relative to a predefined forward direction of the body provides information as to the direction the user is facing.

In a further preferred embodiment, emergency situation detectors are provided to two or more persons in a team. The signals from different members of the team can be compared to determine who is the closest to an event. For example the intensity of an audio signal as received from two different users can be compared to determine who was the closest to an explosion. The team can then be instructed accordingly to deal with the situation.

As an alternative, the physical signal can be compared with a detector of the surroundings, for example a detector located on the wall of the aircraft. Thus vibrations due to the aircraft can be discounted.

In one embodiment, data is stored for a predetermined time in a stack, for example a FIFO stack. The size of the stack may be a given amount of data, or may be a given amount of time, or some other factor as preferred. In the event of the detection of an emergency situation, all of the data currently in the stack is saved, so as to allow subsequent analysis. The stack embodiment is useful because it makes available information from directly before the emergency, often extremely useful in any investigation.

Embodiments of the present invention may use a private communication channel. In one embodiment the equipment located on the user has a short range radio transmitter receiver and a corresponding transmitter receiver is located over a telephone socket. The device at the telephone socket includes an automatic dialer which makes a connection with the controller. For greater range the device at the user may transmit to a repeater which then transmits over a greater range. One embodiment of the repeater may be located at a convenient nearby power socket. Another embodiment may be located on the person. Other embodiments may make use of existing channels such as the cellular network. Yet other embodiments may comprise universal communicators which make use of public networks if detected and use their own channel of communication otherwise.

According to a further embodiment a system comprises rule based logic and one or more body sensors for location on the subject. The subject is expected to follow certain behavioral rules, for example a guard patrols by walking around within a certain area. If he were to run or lie down it would be apparent that an abnormal situation may have arisen. Thus the sensor is usable in combination with the rule based logic to detect non-compliance with the behavioral rules, to indicate an abnormal situation and if necessary to set off an alarm or otherwise summon help. It will be clear that the more independent sensors are used the more reliable the determination can be.

In other circumstances, a guard may be expected to run and lie down to observe suspicious circumstances. In such a case the system may riot react under such circumstances, but may await an additional indication such as an impact or the sound of an explosion, or signs or rolling or the like or an indication of an impact prior to the guard lying/falling down and having his physiological ridings change, which may indicate that the guard is under attack.

In a preferred embodiment, the detectors are programmable. The rules can be changed for different users or for allowing the same device to given to different users having different requirements. The device can also be dynamically programmable according to parameters it is able to detect. Thus it may be able to use detected locations to change between different sets of rules. Or as another example, a device programmed for use by a fireman may change the rules it is using depending on the temperature it detects. In a further example the change of rules may be carried out on line, for example over a radio connection.

A position or location detector may be used in combination with the above system and the rules preferably define location based behaviors.

When used for virtual reality, the main interest is the position or motion detection. However the physiological detection can provide a mood indication.

In the preferred embodiment the signals are translated into the motion of a three-dimensional animated figure on the screen. Reference is now made to FIG. 3 which shows FIG. 30 that uses the signals to replicate the mood and the motion of the subject. The figure is a simplified figure and may be a preconfigured animation.

Thus in emergency situation detection a monitoring party has an intuitive user friendly indication of the state of persons he is monitoring, and in animation and games the animated character moves in accordance with the motions of the actor or player but without needing expensive detection equipment or large scale processing ability.

If the subject being monitored is himself watching the screen then the animated character provides an interactive feedback to the subject. It is possible to carry out recreational and physical excercises and training with immediate and personalized feedback. A subject can be warned if he is not carrying out an exercise correctly or is carrying it out in a dangerous manner. This may be determined by comparing the movement with a predetermined program of movements or comparing the movements with a feature on the virtual reality screen. Furthermore movement of a ball or the direction of a virtual gun can be monitored in combination with the movement of the subject to decide whether a participant in a game has scored points or is killed or the like.

The system can monitor for sounds and the like for emotional content, for example laughter, crying and the like. Likewise the system can monitor the physiological signals for emotional cues. The emotional cues are then transferred to the animated figure.

As a further example if something happens to a subject being monitored, such as him being attacked, the animated figure clearly shows what is happening or what has happened to him at a particular time, since the information can be stored and replayed.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

It will be appreciated by persons skilled in the art that the present invention is not limited to what has been particularly shown and described hereinabove. Rather the scope of the present invention is defined by the appended claims and includes both combinations and subcombinations of the various features described hereinabove as well as variations and modifications thereof which would occur to persons skilled in the art upon reading the foregoing description.

1. Emergency situation detection apparatus comprising:
a stress input unit for receiving body stress information from a subject,
a physical input unit for receiving body physical reaction data from said subject,
a comparator unit, associated with said stress input unit and said physical input unit, for comparing stress level information and physical reaction data, to detect substantially simultaneous stress level change and a physical reaction in said subject,
said apparatus being operable to threshold said simultaneous detection to infer the presence of an emergency situation and to enter an alarm state.

2. Emergency situation detection apparatus according to claim 1, wherein said thresholding is a thresholding of said changes.

3. Emergency situation detection apparatus according to claim 1, configured for attachment to said subject.

4. Emergency situation detection apparatus according to claim 1, configured for attachment to the trunk region of a user, above the hip region.

5. Emergency situation detection apparatus according to claim 1, wherein said stress level information comprises pulse rate information.

6. Emergency situation detection apparatus according to claim 1, wherein said stress level information comprises breathing rate information.

7. Emergency situation detection apparatus according to claim 1 wherein said stress level information is sweat level information.

8. Emergency situation detection apparatus according to claim 1, configured to interpret extremely low stress level information as said subject being in a state of sleep.

9. Emergency situation detection apparatus according to claim 1, wherein said physical input unit comprises an inclination detector affixed to said subject.

10. Emergency situation detection apparatus according to claim 1, wherein said physical input unit comprises an accelerometer.

11. Emergency situation detection apparatus according to claim 1, wherein said input unit is responsive to transmitter units placed on said subjects.

12. Emergency situation detection apparatus according to claim 1, wherein said alarm state comprises automatic opening of a communication channel to a central controller.

13. Emergency situation detection apparatus according to claim 1, wherein said alarm state comprises automatic opening of an audio channel to a central controller.

14. Emergency situation detection apparatus according to claim 13, wherein an end of said audio channel is located on said subject.

15. Emergency situation detection apparatus according to claim 1, wherein said alarm state comprises automatic opening of a video link to a central controller.

16. Emergency situation detection apparatus according to claim 1, said alarm state being additionally triggerable by at least one of an instability monitor, and a loud sound monitor.

17. Emergency situation detection apparatus according to claim 1, sized and configured for mounting unobtrusively on a subject.

18. Emergency situation detection apparatus according to claim 1, further comprising location detection functionality for determining a location, said apparatus further being configured to report said location.

19. Emergency situation detection apparatus according to claim 18, wherein said location detection functionality is one of a group comprising a GPS detector and a triangulation system.

20. Emergency situation detector according to claim 1, further comprising a direction sensor, said direction sensor comprising a compass needle and functionality for measuring an angle of said compass needle in relation to a reference.

21. Emergency situation detector according to claim 1, associated with a memory stack for storing a predetermined amount of immediately preceding data, said detector being configured to save all data in said stack upon entry into said alarm state.

22. Emergency situation detection method comprising:
receiving body stress level information from a subject,
receiving body physical reaction data from said subject,
comparing stress level information and physical reaction data, to detect substantially simultaneous stress level change and physical reaction in said subject, and
thresholding said simultaneous detection to infer the presence of an emergency situation and to enter an alarm state.

23. The method of claim 22, wherein said thresholding comprises thresholding of rates of change.

24. The method of claim 22, wherein said stress level information comprises pulse rate information.

25. The method of claim 22, wherein said stress level information is breathing rate information.

26. The method of claim 22 wherein said stress level information is sweat level information.

27. The method of claim 22 wherein said physical reaction data is body angular inclination data.

28. The method of claim 22, wherein said physical reaction data is body acceleration data.

29. The method of claim 22, comprising being responsive to transmitter units placed on said subject.

30. The method of claim 22, wherein said alarm state comprises automatic opening of a radio link to a central controller.

31. The method of claim 22, wherein said alarm state comprises automatic opening of a video link to a central controller.

32. A system comprising rule based logic and at least one body sensor for location on a subject, the subject being expected to follow certain behavioral rules, said at least one sensor being usable in combination with said rule based logic to detect non-compliance with said behavioral rules, thereby to indicate an abnormal situation.

33. The system of claim 32, further comprising at least a second body sensor usable in combination with said behavioral rules.

34. The system of claim 32, wherein said events include said user sleeping or dozing when he is not expected to.

35. The system of claim 32, wherein said events include said user walking when expected to be stationary or being stationary when expected to be walking.

36. The system of claim 32, wherein said behavioral rules define expected attitudes of user body positions.

37. The system of claim 32, wherein said behavioral rules define places where said user is expected to be located and where said user is expected not to be located.

38. The system of claim 32, wherein said behavioral rules include expected behaviors following major impacts.

39. The system of claim 32, further comprising a location detection device and wherein said rule based logic contains rules based on location.

40. The system of claim 32, being programmable to allow dynamic changing of said rules.

41. A direction sensor for mounting on a mobile body, said direction sensor comprising a compass needle and functionality for measuring an angle of said compass needle in relation to a reference, said direction sensor being configured for mounting in orientation fixed manner on said mobile body.

42. The direction sensor of claim 41, wherein said mobile body is a person, said direction sensor being configured for mounting on a part of said person indicative of a direction that said person is facing.

43. Emergency situation detection apparatus comprising:
a physiological input unit for receiving body physiological information from a subject,
a physical input unit for receiving body physical reaction data from said subject,
a logic unit, associated with said physiological input unit and said physical input unit, for applying at least one logical operation simultaneously to said physiological information and said physical information, to infer the presence of an emergency situation and to enter an alarm state.

44. Emergency situation detection apparatus according to claim 43, configured for attachment to said subject.

45. Emergency situation detection apparatus according to claim 44, configured for attachment to the trunk region of a user, above the hip region.

46. Emergency situation detection apparatus according to claim 43, wherein said physiological level information comprises pulse rate information.

47. Emergency situation detection apparatus according to claim 43, wherein said physiological level information comprises breathing rate information.

48. Emergency situation detection apparatus according to claim 43 wherein said physiological level information is sweat level information.

49. Emergency situation detector according to claim 43, wherein said physiological information is data indicating whether a user is asleep.

50. Emergency situation detection apparatus according to claim 43, wherein said physical input unit comprises an inclination detector affixed to said subject.

51. Emergency situation detection apparatus according to claim 43, wherein said physical input unit comprises an accelerometer.

52. Emergency situation detection apparatus according to claim 43, wherein said input unit is responsive to transmitter units placed on said subjects.

53. Emergency situation detection apparatus according to claim 43, wherein said alarm state comprises automatic opening of a communication channel to a central controller.

54. Emergency situation detection apparatus according to claim 43, wherein said alarm state comprises automatic opening of an audio channel to a central controller.

55. Emergency situation detection apparatus according to claim 54, wherein an end of said audio channel is located on said subject.

56. Emergency situation detection apparatus according to claim 43, wherein said alarm state comprises automatic opening of a video link to a central controller.

57. Emergency situation detection apparatus according to claim 43, said alarm state being additionally triggerable by at least one of an instability monitor, and a loud sound monitor.

58. Emergency situation detection apparatus according to claim 43, sized and configured for mounting unobtrusively on a subject.

59. Emergency situation detection apparatus according to claim 43, further comprising location detection functionality for determining a location, said apparatus further being configured to report said location.

60. Emergency situation detection apparatus according to claim 59, wherein said location detection functionality is one of a group comprising a GPS detector and a triangulation system.

61. Emergency situation detector according to claim 43, further comprising a direction sensor, said direction sensor comprising a compass needle and functionality for measuring an angle of said compass needle in relation to a reference.

62. Emergency situation detector according to claim 43, associated with a memory stack for storing a predetermined amount of immediately preceding data, said detector being configured to save all data in said stack upon entry into said alarm state.

63. Emergency situation detector according to claim 1 wherein said indication of an alarm state comprises any of an impact, an impact causing a subject to falling down, and an impact causing physiological readings to change.

64. Emergency situation detector according to claim 22 wherein said indication of an alarm state comprises any of an impact, an impact causing a subject to falling down, and an impact causing physiological readings to change.

65. Emergency situation detector according to claim 32 wherein said indication of an alarm state comprises any of an impact, an impact causing a subject to fall down, and an impact causing physiological readings to change.

66. Emergency situation detector according to claim 43 wherein said indication of an alarm state comprises any of an impact, an impact causing a subject to fall down, and an impact causing physiological readings to change.

67. Emergency situation detection apparatus according to claim 1, wherein an additional detector is located on a wall of a vehicle carrying a subject, so as to cancel out vibrations of said vehicle.

68. Central co-ordination unit for controlling a plurality of remotely located emergency situation detectors, each detector comprising:
a stress input unit for receiving body stress information from a subject,
a physical input unit for receiving body physical reaction data from said subject,
a comparator unit, associated with said stress input unit and said physical input unit, for comparing stress level information and physical reaction data, to detect substantially simultaneous stress level change and a physical reaction in said subject,
said apparatus being operable to threshold said simultaneous detection to infer the presence of an emergency situation and to enter an alarm state,
said co-ordination unit comprising an alarm unit for indicating that one of said detectors has entered an alarm state and a contact unit for contacting and providing a location of said one of said detectors having entered said alarm state.

69. Central co-ordination unit for controlling a plurality of remotely located emergency situation detectors, each detector comprising:
a physical input unit for receiving body physical reaction data from said subject,
said apparatus being operable to threshold said detection to infer the presence of an emergency situation and to enter an alarm state,
said co-ordination unit comprising an alarm unit for indicating that one of said detectors has entered an alarm state and a contact unit for contacting and providing a location of said one of said detectors having entered said alarm state.

70. Emergency situation detection apparatus according to claim 43, wherein an additional detector is located on a wall of a vehicle carrying a subject, so as to cancel out vibrations of said vehicle.
